(19) United States
(12) Patent Application Publication
(10) Pub. No.: US 2025/0108163 A1
(43) Pub. Date: Apr. 3, 2025

(54) DRUG DELIVERY DEVICE

(71) Applicant: ALTEK BIOTECHNOLOGY CORPORATION, Hsinchu City (TW)

(72) Inventors: Yi Chu, Hsinchu City (TW); Hao-Kuang Ku, Hsinchu City (TW); Yu-Cheng Huang, Hsinchu City (TW)

(21) Appl. No.: 18/895,333

(22) Filed: Sep. 24, 2024

Related U.S. Application Data
(60) Provisional application No. 63/540,944, filed on Sep. 28, 2023.

(51) Int. Cl. A61M 5/142; G16H 20/17

(52) U.S. Cl. CPC A61M 5/14248; G16H 20/17

(57) ABSTRACT

A drug delivery device includes a casing, a drug infusion member, an injection member, a sensing module and a processing unit. The drug infusion member is disposed in the casing. The injection member is disposed in the casing and connected to the drug infusion member. The sensing module is disposed in the casing and configured to sense statuses of the drug infusion member and the injection member. The processing unit is disposed in the casing and coupled to the sensing module. When the processing unit determines that a medical fluid is filled in the drug infusion member in response to the sensing module, the processing unit selectively controls the injection member to be exposed from the casing.

### CROSS REFERENCE TO RELATED APPLICATIONS

This application claims the benefit of U.S. Provisional Application No. 63/540,944, filed on Sep. 28, 2023. The content of the application is incorporated herein by reference.

### BACKGROUND OF THE INVENTION

### 1. Field of the Invention

The invention relates to a drug delivery device and, more particularly, to a drug delivery device capable of ensuring injection safety.

### 2. Description of the Prior Art

A drug delivery device is used to inject a medical fluid into a human body based on a medical behavior. During an injection process of the drug delivery device, some operational actions may influence injection safety of a human body, e.g. whether a medical fluid is filled in an infusion tube, whether the drug delivery device is placed on the human body, whether an injection needle is exposed from or hidden in the drug delivery device, whether air bubble exists in the infusion tube, and so on. Therefore, how to ensure injection safety has become a significant design issue.

### SUMMARY OF THE INVENTION

The invention provides a drug delivery device capable of ensuring injection safety, so as to solve the aforesaid problems.

According to an embodiment of the invention, a drug delivery device comprises a casing, a drug infusion member, an injection member, a sensing module and a processing unit. The drug infusion member is disposed in the casing. The injection member is disposed in the casing and connected to the drug infusion member. The sensing module is disposed in the casing and configured to sense statuses of the drug infusion member and the injection member. The processing unit is disposed in the casing and coupled to the sensing module. When the processing unit determines that a medical fluid is filled in the drug infusion member in response to the sensing module, the processing unit selectively controls the injection member to be exposed from the casing.

In an embodiment, when the processing unit determines that the medical fluid is exhausted from the drug infusion member in response to the sensing module, the processing unit controls the injection member to be hidden in the casing.

In an embodiment, the injection member is movably disposed in the casing, and the processing unit controls the injection member to move with respect to the casing to be exposed from or hidden in the casing.

In an embodiment, the casing comprises a shielding member, the shielding member is movably disposed with respect to the injection member, and the processing unit controls the shielding member to move with respect to the injection member, such that the injection member is exposed from or hidden in the shielding member.

In an embodiment, the drug delivery device further comprises a safety detection member movably disposed in the casing. The sensing module is configured to sense a status of the safety detection member. When the processing unit determines that the safety detection member is hidden in the casing in response to the sensing module, the processing unit controls the medical fluid to be infused into the drug infusion member first and then controls the injection member to be exposed from the casing.

In an embodiment, when the processing unit determines that the safety detection member is exposed from the casing in response to the sensing module, the processing unit controls the injection member to be hidden in the casing.

In an embodiment, when the casing is placed on an object and the safety detection member faces the object, the object forces the safety detection member to be hidden in the casing.

In an embodiment, when the casing moves away from the object, the safety detection member is automatically exposed from the casing.

In an embodiment, the safety detection member surrounds the injection member.

In an embodiment, when the casing moves away from the object, the safety detection member is automatically exposed from the casing and self-locked.

In an embodiment, the sensing module comprises at least one optical sensor configured to sense an amount of light related to at least one of the drug infusion member, the injection member and the safety detection member.

In an embodiment, the sensing module comprises an optical sensor, and the drug infusion member, the safety detection member and the injection member are arranged along an optical axis of the optical sensor.

In an embodiment, the sensing module comprises two optical sensors, one of the drug infusion member, the safety detection member and the injection member is arranged along an optical axis of one of the two optical sensors, and the other two of the drug infusion member, the safety detection member and the injection member are arranged along an optical axis of the other one of the two optical sensors.

In an embodiment, the drug delivery device further comprises a motor disposed in the casing, coupled to the processing unit and configured to infuse the medical fluid into the drug infusion member. The sensing module is configured to sense a current number of revolutions of the motor. When the current number of revolutions is smaller than or equal to a first predetermined number of revolutions, the processing unit determines that the drug delivery device is in an injection-in-progress stage. When the current number of revolutions is larger than the first predetermined number of revolutions, the processing unit determines that the drug delivery device is in a ready-to-stop stage.

In an embodiment, when the drug delivery device is in the injection-in-progress stage and the sensing module senses that at least one air bubble exists in the drug infusion member, the processing unit determines that the drug infusion member is leaking.

In an embodiment, the drug delivery device further comprises an alarm unit disposed in the casing and coupled to the processing unit. When the processing unit determines that the drug infusion member is leaking, the processing unit controls the alarm unit to send out an alarm.

In an embodiment, when the drug delivery device is in the ready-to-stop stage and the sensing module senses that at least one air bubble exists in the drug infusion member, the processing unit determines whether a percentage of the at least one air bubble to the medical fluid within a time period is larger than a threshold. When the percentage of the at least one air bubble to the medical fluid within the time period is larger than the threshold, the processing unit determines that an injection process of the drug delivery device has been completed and controls the motor to stop.

In an embodiment, when the percentage of the at least one air bubble to the medical fluid within the time period is smaller than or equal to the threshold, the motor continues to operate until the current number of revolutions reaches a second predetermined number of revolutions. The second predetermined number of revolutions is larger than the first predetermined number of revolutions.

In an embodiment, the sensing module comprises two optical sensors, the drug infusion member is arranged along an optical axis of one of the two optical sensors, and a rotating portion of the motor is arranged along an optical axis of the other one of the two optical sensors.

According to an embodiment of the invention, a drug delivery device comprises a casing, a drug infusion member, a motor, a sensing module and a processing unit. The drug infusion member is disposed in the casing. The motor is disposed in the casing and configured to infuse a medical fluid into the drug infusion member. The sensing module is disposed in the casing and configured to sense a status of the drug infusion member and sense a current number of revolutions of the motor. The processing unit is disposed in the casing and coupled to the motor and the sensing module. When the current number of revolutions is larger than a first predetermined number of revolutions, the processing unit determines that the drug delivery device is in a ready-to-stop stage. When the drug delivery device is in the ready-to-stop stage and the sensing module senses that at least one air bubble exists in the drug infusion member, the processing unit determines whether a percentage of the at least one air bubble to the medical fluid within a time period is larger than a threshold. When the percentage of the at least one air bubble to the medical fluid within the time period is larger than the threshold, the processing unit determines that an injection process of the drug delivery device has been completed and controls the motor to stop.

In an embodiment, when the percentage of the at least one air bubble to the medical fluid within the time period is smaller than or equal to the threshold, the motor continues to operate until the current number of revolutions reaches a second predetermined number of revolutions. The second predetermined number of revolutions is larger than the first predetermined number of revolutions.

In an embodiment, when the current number of revolutions is smaller than or equal to the first predetermined number of revolutions, the processing unit determines that the drug delivery device is in an injection-in-progress stage. When the drug delivery device is in the injection-in-progress stage and the sensing module senses that the at least one air bubble exists in the drug infusion member, the processing unit determines that the drug infusion member is leaking.

In an embodiment, the drug delivery device further comprises an alarm unit disposed in the casing and coupled to the processing unit. When the processing unit determines that the drug infusion member is leaking, the processing unit controls the alarm unit to send out an alarm.

In an embodiment, the sensing module comprises two optical sensors, the drug infusion member is arranged along an optical axis of one of the two optical sensors, and a rotating portion of the motor is arranged along an optical axis of the other one of the two optical sensors.

As mentioned in the above, the drug delivery device of the invention utilizes the sensing module to sense statuses of the drug infusion member, the injection member and/or the safety detection member, so as to identify corresponding operational actions individually or in combination (e.g. whether the medical fluid is filled in the drug infusion member, whether the drug delivery device is placed on a human body, whether the injection member is exposed from or hidden in the drug delivery device, and/or whether at least one air bubble exists in the drug infusion member), such that the processing unit of the drug delivery device may perform corresponding safety mechanism to ensure injection safety of the human body. Furthermore, the sensing module may sense the current number of revolutions of the motor, which is configured to infuse the medical fluid into the drug infusion member, so as to determine that the drug delivery device is in an injection-in-progress stage or a ready-to-stop stage. Then, the processing unit of the drug delivery device may determine whether the drug infusion member is leaking in the injection-in-progress stage or determine whether the injection process of the drug delivery device has been completed in the ready-to-stop stage, so as to perform corresponding safety mechanism to ensure injection safety of the human body.

These and other objectives of the present invention will no doubt become obvious to those of ordinary skill in the art after reading the following detailed description of the preferred embodiment that is illustrated in the various figures and drawings.

### BRIEF DESCRIPTION OF THE DRAWINGS

FIG. 1 is a schematic view illustrating a drug delivery device according to an embodiment of the invention.

FIG. 2 is a schematic view illustrating a casing of the drug delivery device being placed on an object.

FIG. 3 is a schematic view illustrating a medical fluid being filled in a drug infusion member and an injection member being exposed from the casing.

FIG. 4 is a schematic view illustrating a drug delivery device according to an embodiment of the invention.

FIG. 5 is a schematic view illustrating the medical fluid being filled in the drug infusion member and the injection member being exposed from the casing.

FIG. 6 is a schematic view illustrating a drug delivery device according to an embodiment of the invention.

FIG. 7 is a schematic view illustrating the casing of the drug delivery device being placed on the object.

FIG. 8 is a schematic view illustrating the medical fluid being filled in the drug infusion member and the injection member being exposed from the casing.

FIG. 9 is a schematic view illustrating a drug delivery device according to an embodiment of the invention.

FIG. 10 is a schematic view illustrating the casing of the drug delivery device being placed on the object.

FIG. 11 is a schematic view illustrating the medical fluid being filled in the drug infusion member and the injection member being exposed from the casing.

FIG. 12 is a schematic view illustrating the safety detection member is automatically exposed from the casing and self-locked.

FIG. 13 is a schematic view illustrating a drug delivery device according to an embodiment of the invention.

FIG. 14 is a schematic view illustrating a drug delivery device according to an embodiment of the invention.

### DETAILED DESCRIPTION

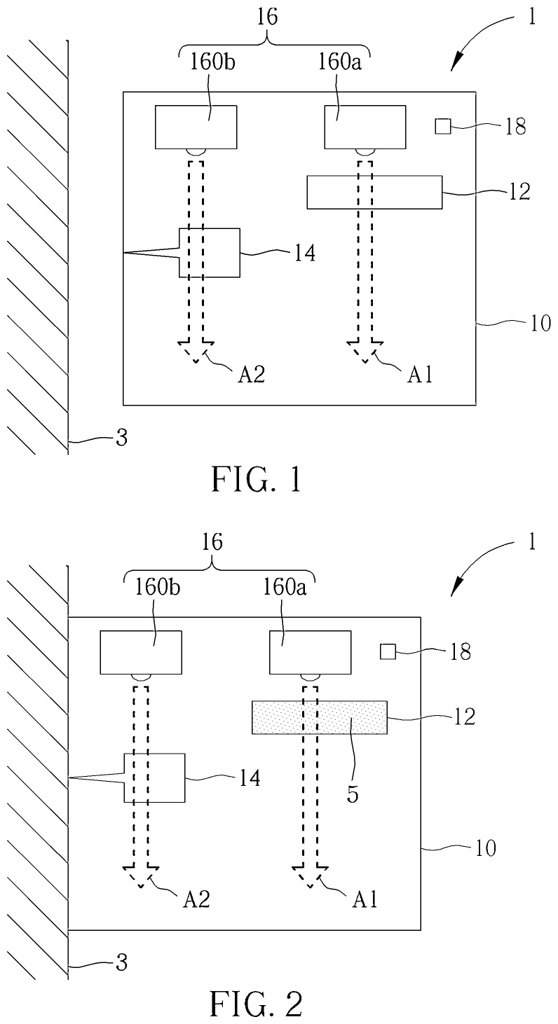

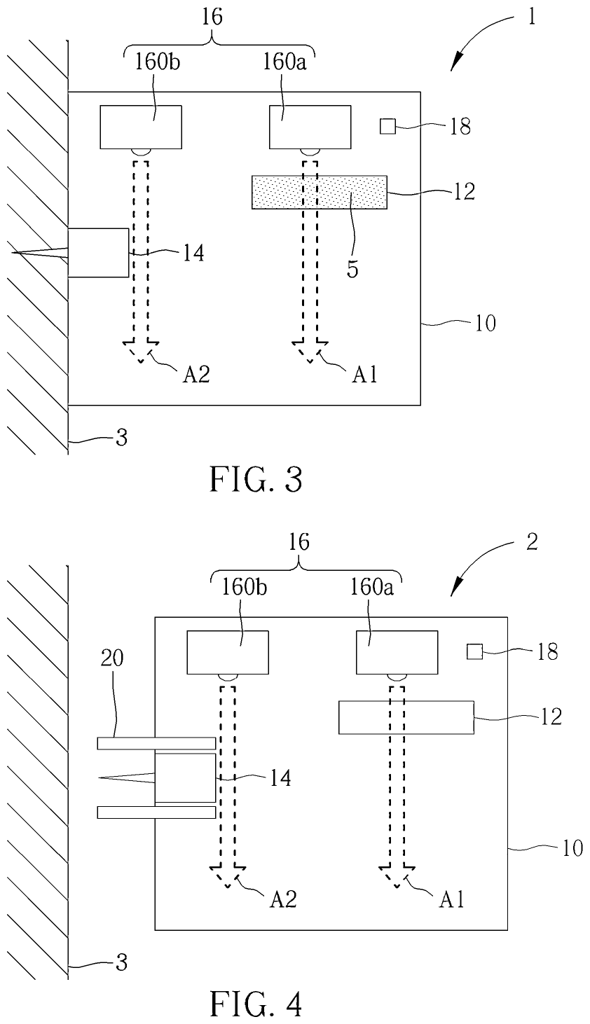

Referring to FIGS. 1 to 3, FIG. 1 is a schematic view illustrating a drug delivery device 1 according to an embodiment of the invention, FIG. 2 is a schematic view illustrating a casing 10 of the drug delivery device 1 being placed on an object 3, and FIG. 3 is a schematic view illustrating a medical fluid 5 being filled in a drug infusion member 12 and an injection member 14 being exposed from the casing 10.

As shown in FIGS. 1 to 3, the drug delivery device 1 comprises a casing 10, a drug infusion member 12, an injection member 14, a sensing module 16 and a processing unit 18, wherein the drug infusion member 12, the injection member 14, the sensing module 16 and the processing unit 18 are disposed in the casing 10. The injection member 14 is connected to the drug infusion member 12, such that a medical fluid 5 (as shown in FIG. 3) filled in the drug infusion member 12 can be delivered to the injection member 14. In this embodiment, a motor (not shown) may be disposed in the casing 10 and configured to infuse the medical fluid 5 into the drug infusion member 12. It should be noted that how the motor infuses the medical fluid 5 into the drug infusion member 12 is well known by one skilled in the art, so it will not be depicted in detail herein. In this embodiment, the drug infusion member 12 may be a drug infusion tube or the like, and the injection member 14 may be an injection needle or the like.

The sensing module 16 is configured to sense statuses of the drug infusion member 12 and the injection member 14. In this embodiment, the sensing module 16 may comprise two optical sensors 160a, 160b, wherein one of the drug infusion member 12 and the injection member 14 is arranged along an optical axis A1 of one of the two optical sensors 160a, 160b, and the other one of the drug infusion member 12 and the injection member 14 is arranged along an optical axis A2 of the other one of the two optical sensors 160a, 160b. As shown in FIGS. 1 to 3, the drug infusion member 12 may be arranged along the optical axis A1 of the optical sensor 160a, and the injection member 14 may be arranged along the optical axis A2 of the optical sensor 160b. The optical sensors 160a, 160b may be, but are not limited to, reflective photoelectric sensors. In practical applications, the reflective photoelectric sensor may consist of a light emitting diode (LED) that emits light and a detector that receives the reflected light from an object. It should be noted that the principle of reflective photoelectric sensor is well known by one skilled in the art, so it will not be depicted in detail herein.

As shown in FIGS. 1 and 2, when the drug infusion member 12 is not filled with the medical fluid 5 or filled with the medical fluid 5, the optical sensor 160a may sense different amount of reflected light relative to the drug infusion member 12 for the processing unit 18 to distinguish different statuses of the drug infusion member 12. Similarly, as shown in FIGS. 2 and 3, when the injection member 14 is hidden in the casing 10 or exposed from the casing 10, the optical sensor 160b may sense different amount of reflected light relative to the injection member 14 for the processing unit 18 to distinguish different statuses of the injection member 14. It should be noted that the amount of reflected light may be obtained by various techniques, such as: 1) choice of materials or elements (e.g. liquid), including material chemistry, color, opacity, surface texture and/or surface chemistry; 2) stacking order of the elements for reflection; 3) distance of the elements from the sensor; and 4) total or partial presence or absence of the functional structures/elements in the sensing zone/range.

The processing unit 18 is coupled to the sensing module 16. In this embodiment, the processing unit 18 may be coupled to the sensing module 16 through a circuit layout (e.g. circuit board, not shown). In practical applications, the processing unit 18 may be a processor or a controller with data processing function.

Before the drug delivery device 1 starts to perform an injection process, the drug infusion member 12 is not filled with any medical fluid and the injection member 14 is hidden in the casing 10, as shown in FIG. 1. When a user wants to use the drug delivery device 1 to perform an injection process, the user may place the casing 10 of the drug delivery device 1 on an object 3 (e.g. human body) first, as shown in FIG. 2. Then, a medical fluid 5 may be driven by the aforesaid motor to be filled in the drug infusion member 12. At this time, the injection member 14 is still hidden in the casing 10 to prevent the injection member 14 from harming the user. When the processing unit 18 determines that the medical fluid 5 is filled in the drug infusion member 12 in response to the sensing module 16, the processing unit 18 will selectively control the injection member 14 to be exposed from the casing 10. As shown in FIG. 3, since the medical fluid 5 is filled in the drug infusion member 12, the processing unit 18 controls the injection member 14 to be exposed from the casing 10, so as to perform the injection process. For further explanation, when the injection member 14 is exposed from the casing 10, the injection member 14 will penetrate into the object 3, such that the medical fluid 5 in the drug infusion member 12 can be delivered to the object 3 through the injection member 14.

When the processing unit 18 determines that the medical fluid 5 is exhausted from the drug infusion member 12 in response to the sensing module 16, it means that the injection process of the drug delivery device 1 has been completed. At this time, the processing unit 18 will control the injection member 14 to be hidden in the casing 10. Accordingly, when the user removes the casing 10 of the drug delivery device 1 from the object 3, the user will not be harmed by the injection member 14.

In this embodiment, the injection member 14 is movably disposed in the casing 10. Thus, the processing unit 18 can control the injection member 14 to move with respect to the casing 10 to be exposed from or hidden in the casing 10, as shown in FIGS. 2 and 3. In practical applications, the injection member 14 may be driven to move through a driving mechanism (not shown) including a motor and a transmission structure (e.g. linkage rod, gear and so on).

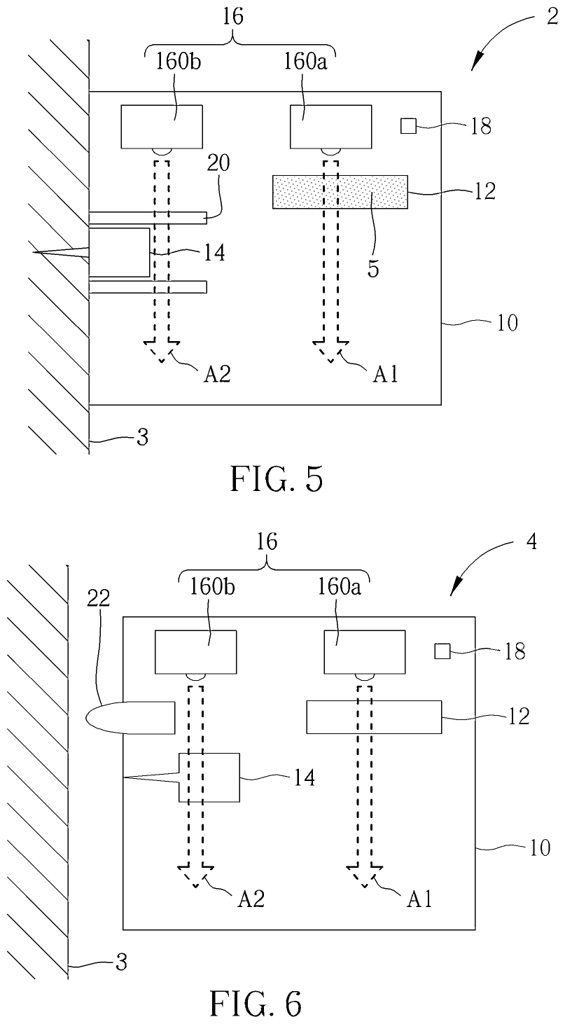

Referring to FIGS. 4 and 5, FIG. 4 is a schematic view illustrating a drug delivery device 2 according to an embodiment of the invention, and FIG. 5 is a schematic view illustrating the medical fluid 5 being filled in the drug infusion member 12 and the injection member 14 being exposed from the casing 10.

The main difference between the drug delivery device 2 and the aforesaid drug delivery device 1 is that the casing 10 of the drug delivery device 2 comprises a shielding member 20, as shown in FIGS. 4 and 5. The shielding member 20 is movably disposed with respect to the injection member 14. Thus, the processing unit can control the shielding member 20 to move with respect to the injection member 14, such that the injection member 14 is exposed from or hidden in the shielding member 20 of the casing 10. In practical applications, the shielding member 20 may be driven to move through a driving mechanism (not shown) including a motor and a transmission structure (e.g. linkage rod, gear and so on).

In this embodiment, the shielding member 20 may be arranged along the optical axis A2 of the optical sensor 160b. Thus, when the shielding member 20 moves to make the injection member 14 hide in the shielding member 20 or expose from the shielding member 20, the optical sensor 160b may sense different amount of reflected light relative to the shielding member 20 for the processing unit 18 to distinguish different statuses of the injection member 14.

Before the drug delivery device 2 starts to perform an injection process, the drug infusion member 12 is not filled with any medical fluid and the injection member 14 is hidden in the shielding member 20 of the casing 10, as shown in FIG. 4. When a user wants to use the drug delivery device 2 to perform an injection process, the user may place the casing 10 of the drug delivery device 2 on the object 3 (e.g. human body) first. Then, the medical fluid 5 may be driven by the aforesaid motor to be filled in the drug infusion member 12. At this time, the injection member 14 is still hidden in the shielding member 20 of the casing 10 to prevent the injection member 14 from harming the user. When the processing unit 18 determines that the medical fluid 5 is filled in the drug infusion member 12 in response to the sensing module 16, the processing unit 18 will selectively control the injection member 14 to be exposed from the casing 10. For further explanation, the processing unit 18 will control the shielding member 20 to move to make the injection member 14 expose from the shielding member 20 of the casing 10. As shown in FIG. 5, the injection member 14 will penetrate into the object 3, such that the medical fluid 5 in the drug infusion member 12 can be delivered to the object 3 through the injection member 14.

When the processing unit 18 determines that the medical fluid 5 is exhausted from the drug infusion member 12 in response to the sensing module 16, it means that the injection process of the drug delivery device 2 has been completed. At this time, the processing unit 18 will control the shielding member 20 to move to make the injection member 14 hide in the shielding member 20 of the casing 10. Accordingly, when the user removes the casing 10 of the drug delivery device 2 from the object 3, the user will not be harmed by the injection member 14. In an embodiment, when the shielding member 20 is exposed from the casing 10 after the injection process of the drug delivery device 2 has been completed (as the status shown in FIG. 4), the shielding member 20 may be self-locked by a self-locking mechanism at the same time. The self-locking mechanism may be achieved by the principle of an automatic ball pen or the like, and it is well known by one skilled in the art, so it will not be depicted in detail herein. When the shielding member 20 is self-locked, the shielding member 20 may not be pushed into the casing 10 again, such that the injection member 14 is hidden in the shielding member 20 no matter whether the injection member 14 is exposed from or hidden in the casing 10.

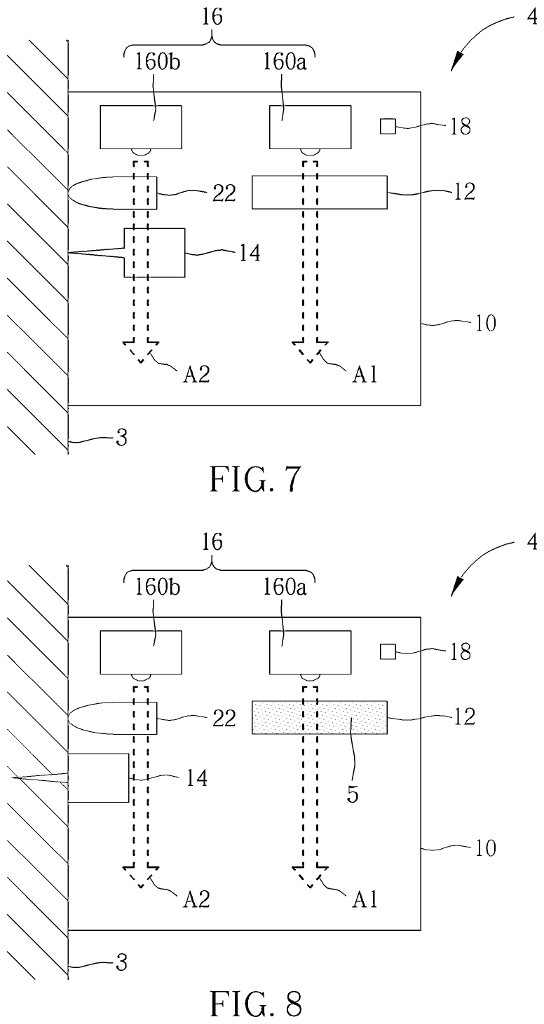

Referring to FIGS. 6 to 8, FIG. 6 is a schematic view illustrating a drug delivery device 4 according to an embodiment of the invention, FIG. 7 is a schematic view illustrating the casing 10 of the drug delivery device 4 being placed on the object 3, and FIG. 8 is a schematic view illustrating the medical fluid 5 being filled in the drug infusion member 12 and the injection member 14 being exposed from the casing 10.

The main difference between the drug delivery device 4 and the aforesaid drug delivery device 1 is that the drug delivery device 4 further comprises a safety detection member 22 movably disposed in the casing 10, as shown in FIGS. 6 to 8. In this embodiment, the safety detection member 22 may be any activation related mechanisms, e.g. device body sensor, container sensor, etc.

In this embodiment, the sensing module 16 is further configured to sense a status of the safety detection member 22, and the safety detection member 22 may be arranged along the optical axis A2 of the optical sensor 160b. Thus, when the safety detection member 22 moves to be hidden in the casing 10 or exposed from the casing 10, the optical sensor 160b may sense different amount of reflected light relative to the safety detection member 22 for the processing unit 18 to distinguish different statuses of the safety detection member 22.

In this embodiment, the drug infusion member 12 is arranged along the optical axis A1 of the optical sensor 160a, and the injection member 14 and the safety detection member 22 are arranged along the optical axis A2 of the optical sensor 160b. However, in another embodiment, one of the drug infusion member 12, the safety detection member 22 and the injection member 14 may be arranged along the optical axis of one of the two optical sensors 160a, 160b, and the other two of the drug infusion member 12, the safety detection member 22 and the injection member 14 may be arranged along the optical axis of the other one of the two optical sensors 160a, 160b. That is to say, the arrangement of the drug infusion member 12, the safety detection member 22 and the injection member 14 relative to the two optical sensors 160a, 160b may be determined according to practical applications.

Before the drug delivery device 4 starts to perform an injection process, the drug infusion member 12 is not filled with any medical fluid, the injection member 14 is hidden in the casing 10, and the safety detection member 22 is exposed from the casing 10, as shown in FIG. 6. When a user wants to use the drug delivery device 4 to perform an injection process, the user may place the casing 10 of the drug delivery device 4 on the object 3 (e.g. human body) first, as shown in FIG. 7. When the casing 10 is placed on the object 3 and the safety detection member 22 faces the object 3, the object 3 forces the safety detection member 22 to be hidden in the casing 10. When the processing unit 18 determines that the safety detection member 22 is hidden in the casing 10 in response to the sensing module 16, the processing unit 18 will control the medical fluid 5 to be infused into the drug infusion member 12 first and then controls the injection member 14 to be exposed from the casing 10, as shown in FIG. 8. At this time, the injection member 14 will penetrate into the object 3, such that the medical fluid 5 in the drug infusion member 12 can be delivered to the object 3 through the injection member 14.

When the medical fluid 5 is exhausted from the drug infusion member 12, the user may remove the casing 10 of the drug delivery device 4 from the object 3. When the casing 10 moves away from the object 3, the safety detection member 22 may be automatically exposed from the casing 10. In this embodiment, an elastic member (e.g. spring, not shown) may be disposed with respect to the safety detection member 22, such that the elastic member may be compressed by the safety detection member 22 and provide an elastic force for returning the safety detection member 22. When the processing unit 18 determines that the safety detection member 22 is exposed from the casing 10 in response to the sensing module 16, the processing unit 18 will control the injection member 14 to be hidden in the casing 10, as shown in FIG. 6. Accordingly, when the user removes the casing 10 of the drug delivery device 4 from the object 3, the user will not be harmed by the injection member 14.

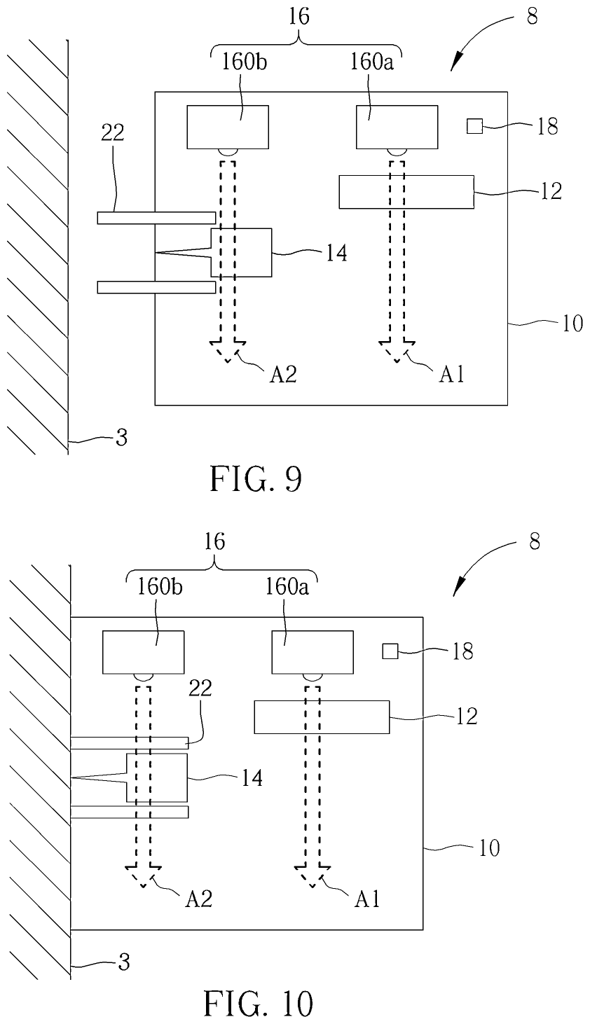

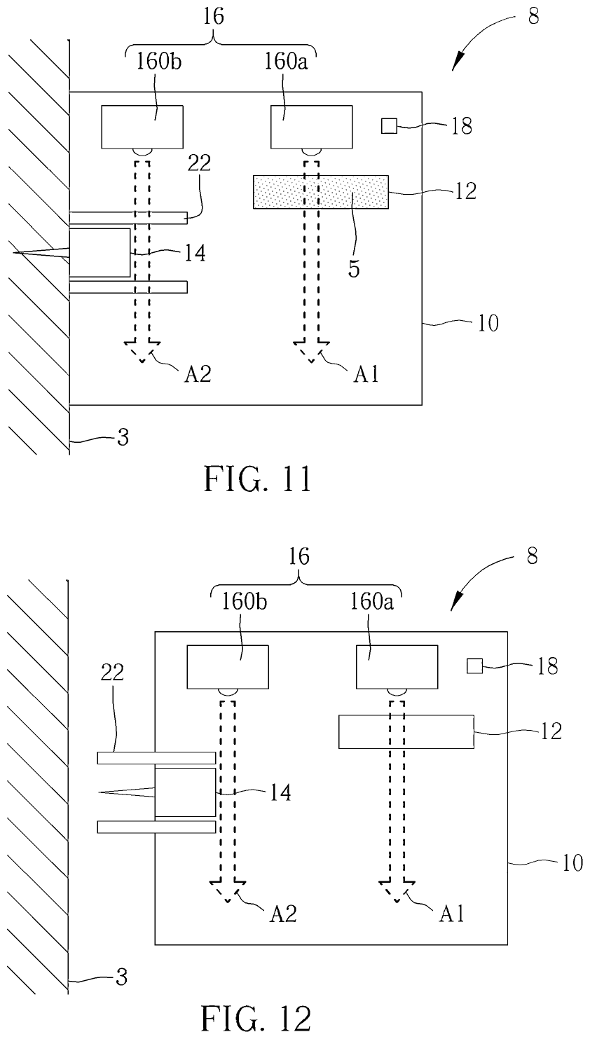

Referring to FIGS. 9 to 12, FIG. 9 is a schematic view illustrating a drug delivery device 8 according to an embodiment of the invention, FIG. 10 is a schematic view illustrating the casing 10 of the drug delivery device 8 being placed on the object 3, FIG. 11 is a schematic view illustrating the medical fluid 5 being filled in the drug infusion member 12 and the injection member 14 being exposed from the casing 10, and FIG. 12 is a schematic view illustrating the safety detection member 22 is automatically exposed from the casing 10 and self-locked.

The main difference between the drug delivery device 8 and the aforesaid drug delivery device 4 is that the safety detection member 22 of the drug delivery device 8 surrounds the injection member 14, as shown in FIGS. 9 to 12. In this embodiment, the safety detection member 22 may be shaped as a hollow tube or hollow block surrounding the injection member 14. In this embodiment, the drug infusion member 12 is arranged along the optical axis A1 of the optical sensor 160a, and the injection member 14 and the safety detection member 22 are arranged along the optical axis A2 of the optical sensor 160b.

Before the drug delivery device 8 starts to perform an injection process, the drug infusion member 12 is not filled with any medical fluid, the injection member 14 is hidden in the casing 10, and the safety detection member 22 is exposed from the casing 10, as shown in FIG. 9. When a user wants to use the drug delivery device 8 to perform an injection process, the user may place the casing 10 of the drug delivery device 8 on the object 3 (e.g. human body) first, as shown in FIG. 10. When the casing 10 is placed on the object 3 and the safety detection member 22 faces the object 3, the object 3 forces the safety detection member 22 to be hidden in the casing 10. When the processing unit 18 determines that the safety detection member 22 is hidden in the casing 10 in response to the sensing module 16, the processing unit 18 will control the medical fluid 5 to be infused into the drug infusion member 12 first and then controls the injection member 14 to be exposed from the casing 10, as shown in FIG. 11. At this time, the injection member 14 will penetrate into the object 3, such that the medical fluid 5 in the drug infusion member 12 can be delivered to the object 3 through the injection member 14.

When the medical fluid 5 is exhausted from the drug infusion member 12, the user may remove the casing 10 of the drug delivery device 8 from the object 3. As shown in FIG. 12, when the casing 10 moves away from the object 3, the safety detection member 22 may be automatically exposed from the casing 10 and self-locked. In this embodiment, an elastic member (e.g. spring, not shown) may be disposed with respect to the safety detection member 22, such that the elastic member may be compressed by the safety detection member 22 and provide an elastic force for returning the safety detection member 22. Furthermore, when the safety detection member 22 is automatically exposed from the casing 10, the safety detection member 22 may be self-locked by a self-locking mechanism at the same time. The self-locking mechanism may be achieved by the principle of an automatic ball pen or the like, and it is well known by one skilled in the art, so it will not be depicted in detail herein. In an embodiment, when the safety detection member 22 is self-locked, the safety detection member 22 may not be pushed into the casing 10 again, such that the injection member 14 is hidden in the safety detection member 22 no matter whether the injection member 14 is exposed from or hidden in the casing 10. Accordingly, when the user removes the casing 10 of the drug delivery device 8 from the object 3, the user will not be harmed by the injection member 14.

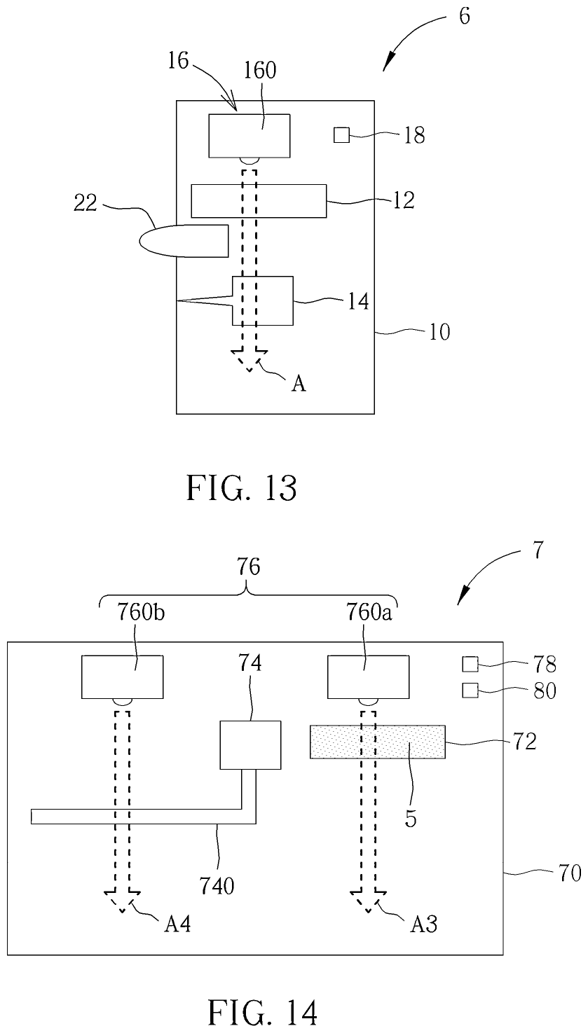

Referring to FIG. 13, FIG. 13 is a schematic view illustrating a drug delivery device 6 according to an embodiment of the invention.

The main difference between the drug delivery device 6 and the aforesaid drug delivery device 4 is that the sensing module 16 of the drug delivery device 6 comprises a single optical sensor 160, and the drug infusion member 12, the safety detection member 22 and the injection member 14 are arranged along an optical axis A of the optical sensor 160.

Therefore, according to the aforesaid embodiments, the sensing module 16 may comprise at least one optical sensor configured to sense an amount of light related to at least one of the drug infusion member 12, the injection member 14 and the safety detection member 22, and it depends on practical applications. Furthermore, the embodiment shown in FIG. 13 may also be applied to the embodiments shown in FIGS. 1 to 5. Still further, in another embodiment, the sensing module 16 may comprise three optical sensors respectively disposed with respect to the drug infusion member 12, the injection member 14 and the safety detection member 22. Moreover, the safety detection member 22 shown in FIG. 13 may be replaced by the safety detection member 22 shown in FIGS. 9 to 12.

Referring to FIG. 14, FIG. 14 is a schematic view illustrating a drug delivery device 7 according to an embodiment of the invention.

As shown in FIG. 14, the drug delivery device 7 comprises a casing 70, a drug infusion member 72, a motor 74, a sensing module 76 and a processing unit 78, wherein the drug infusion member 72, the motor 74, the sensing module 76 and the processing unit 78 are disposed in the casing 70. The motor 74 is configured to infuse a medical fluid 5 into the drug infusion member 72. It should be noted that how the motor 74 infuses the medical fluid 5 into the drug infusion member 72 is well known by one skilled in the art, so it will not be depicted in detail herein. In this embodiment, the drug infusion member 72 may be a drug infusion tube or the like.

The sensing module 76 is configured to sense a status of the drug infusion member 72 and sense a current number of revolutions of the motor 74. In this embodiment, the sensing module 76 may comprise two optical sensors 760a, 760b, wherein the drug infusion member 72 is arranged along an optical axis A3 of one of the two optical sensors 760a, 760b, and a rotating portion 740 of the motor 74 is arranged along an optical axis A4 of the other one of the two optical sensors 760a, 760b. As shown in FIG. 14, the drug infusion member 72 may be arranged along the optical axis A3 of the optical sensor 760a, and the rotating portion 740 of the motor 74 may be arranged along the optical axis A4 of the optical sensor 760b. The optical sensors 760a, 760b may be, but are not limited to, reflective photoelectric sensors. In practical applications, the reflective photoelectric sensor may consist of a light emitting diode (LED) that emits light and a detector that receives the reflected light from an object. It should be noted that the principle of reflective photoelectric sensor is well known by one skilled in the art, so it will not be depicted in detail herein.

When the drug infusion member 72 is not filled with the medical fluid 5 or filled with the medical fluid 5, the optical sensor 760a may sense different amount of reflected light relative to the drug infusion member 72 for the processing unit 78 to distinguish different statuses of the drug infusion member 72. Thus, the processing unit 78 may determine whether at least one air bubble exists in the drug infusion member 72 according to the amount of reflected light relative to the drug infusion member 72. Similarly, when the rotating portion 740 of the motor 74 passes or not pass through the optical axis A4 of the optical sensor 760b, the optical sensor 760b may sense different amount of reflected light relative to the rotating portion 740 to obtain the current number of revolutions of the motor 74. It should be noted that the amount of reflected light may be obtained by various techniques, such as: 1) choice of materials or elements (e.g. liquid), including material chemistry, color, opacity, surface texture and/or surface chemistry; 2) stacking order of the elements for reflection; 3) distance of the elements from the sensor; and 4) total or partial presence or absence of the functional structures/elements in the sensing zone/range.

The processing unit 78 is coupled to the motor 74 and the sensing module 76. In this embodiment, the processing unit 78 may be coupled to the motor 74 and the sensing module 76 through a circuit layout (e.g. circuit board, not shown). In practical applications, the processing unit 78 may be a processor or a controller with data processing function.

When the drug delivery device 7 is used to perform an injection process, the drug delivery device 7 may be in an injection-in-progress stage to inject a major injection volume (e.g. 95% of a target injection volume) and in a ready-to-stop stage to inject a residual injection volume (e.g. 5% of the target injection volume). In the injection-in-progress stage, the motor 74 may be programmed to rotate with a first predetermined number of revolutions, and in ready-to-stop stage, the motor 74 may be programmed to rotate with a second predetermined number of revolutions, wherein the second predetermined number of revolutions is larger than the first predetermined number of revolutions.

Therefore, when the current number of revolutions is larger than the first predetermined number of revolutions, it means that the major injection volume has been injected, such that the processing unit 18 will determine that the drug delivery device 7 is in the ready-to-stop stage. When the drug delivery device 7 is in the ready-to-stop stage and the sensing module 76 senses that at least one air bubble exists in the drug infusion member 72, the processing unit 78 determines whether a percentage of the at least one air bubble to the medical fluid 5 within a time period is larger than a threshold. The threshold may be determined according to practical applications. Then, when the percentage of the at least one air bubble to the medical fluid 5 within the time period is larger than the threshold, it means that the residual injection volume has been almost or completely injected. At this time, the processing unit 18 will determine that the injection process of the drug delivery device 7 has been completed and controls the motor 74 to stop even if the second predetermined number of revolutions has not been reached.

On the other hand, when the percentage of the at least one air bubble to the medical fluid 5 within the time period is smaller than or equal to the threshold, it means that the residual injection volume has not been completely injected. At this time, the motor 74 continues to operate until the current number of revolutions reaches the second predetermined number of revolutions. In general, when the current number of revolutions reaches the second predetermined number of revolutions, the residual injection volume should have been almost or completely injected.

Furthermore, when the current number of revolutions is smaller than or equal to the first predetermined number of revolutions, the processing unit 78 will determine that the drug delivery device 7 is in the injection-in-progress stage. When the drug delivery device 7 is in the injection-in-progress stage and the sensing module 76 senses that the at least one air bubble exists in the drug infusion member 72, the processing unit 78 will determine that the drug infusion member 72 is leaking.

As shown in FIG. 14, the drug delivery device 7 may further comprise an alarm unit 80 disposed in the casing 70 and coupled to the processing unit 78. When the processing unit 78 determines that the drug infusion member 72 is leaking, the processing unit 78 may control the alarm unit 80 to send out an alarm for warning the user, so as to avoid danger during the injection process.

The embodiment shown in FIG. 14 may also be applied to the embodiments shown in FIGS. 1 to 13. The sensing module may comprise at least one optical sensor disposed with respect to the drug infusion member, the injection member, the safety detection member and the motor according to practical applications, and the arrangement of the aforesaid components may also be determined according to practical applications.

It should be noted that, in addition to the number of revolutions of the motor, the invention may also utilize a current injection time to determine that the drug delivery device is in an injection-in-progress stage or in a ready-to-stop stage, wherein the injection time may be counted by a timer. For example, when the current injection time is within a first injection time period, the processing unit may determine that the drug delivery device is in the injection-in-progress stage; and when the current injection time is beyond the first injection time period and within a second injection time period, the processing unit may determine that the drug delivery device is in the ready-to-stop stage.

Therefore, in the ready-to-stop stage, when the residual injection volume has been almost or completely injected, the processing unit will control the motor to stop even if the current injection time is not beyond the second injection time period. On the other hand, when the residual injection volume has not been completely injected, the motor continues to operate until the second injection time period has been reached. In general, when the second injection time period has been reached, the residual injection volume should have been almost or completely injected.

As mentioned in the above, the drug delivery device of the invention utilizes the sensing module to sense statuses of the drug infusion member, the injection member and/or the safety detection member, so as to identify corresponding operational actions individually or in combination (e.g. whether the medical fluid is filled in the drug infusion member, whether the drug delivery device is placed on a human body, whether the injection member is exposed from or hidden in the drug delivery device, and/or whether at least one air bubble exists in the drug infusion member), such that the processing unit of the drug delivery device may perform corresponding safety mechanism to ensure injection safety of the human body. Furthermore, the sensing module may sense the current number of revolutions of the motor, which is configured to infuse the medical fluid into the drug infusion member, so as to determine that the drug delivery device is in an injection-in-progress stage or a ready-to-stop stage. Then, the processing unit of the drug delivery device may determine whether the drug infusion member is leaking in the injection-in-progress stage or determine whether the injection process of the drug delivery device has been completed in the ready-to-stop stage, so as to perform corresponding safety mechanism to ensure injection safety of the human body.

Those skilled in the art will readily observe that numerous modifications and alterations of the device and method may be made while retaining the teachings of the invention. Accordingly, the above disclosure should be construed as limited only by the metes and bounds of the appended claims.

What is claimed is:

1. A drug delivery device comprising:
a casing;
a drug infusion member disposed in the casing;
an injection member disposed in the casing and connected to the drug infusion member;
a sensing module disposed in the casing and configured to sense statuses of the drug infusion member and the injection member; and
a processing unit disposed in the casing and coupled to the sensing module;
wherein, when the processing unit determines that a medical fluid is filled in the drug infusion member in response to the sensing module, the processing unit selectively controls the injection member to be exposed from the casing.

2. The drug delivery device of claim 1, wherein, when the processing unit determines that the medical fluid is exhausted from the drug infusion member in response to the sensing module, the processing unit controls the injection member to be hidden in the casing.

3. The drug delivery device of claim 2, wherein the injection member is movably disposed in the casing, and the processing unit controls the injection member to move with respect to the casing to be exposed from or hidden in the casing.

4. The drug delivery device of claim 2, wherein the casing comprises a shielding member, the shielding member is movably disposed with respect to the injection member, and the processing unit controls the shielding member to move with respect to the injection member, such that the injection member is exposed from or hidden in the shielding member.

5. The drug delivery device of claim 1, further comprising a safety detection member movably disposed in the casing, the sensing module being configured to sense a status of the safety detection member, wherein, when the processing unit determines that the safety detection member is hidden in the casing in response to the sensing module, the processing unit controls the medical fluid to be infused into the drug infusion member first and then controls the injection member to be exposed from the casing.

6. The drug delivery device of claim 5, wherein, when the processing unit determines that the safety detection member is exposed from the casing in response to the sensing module, the processing unit controls the injection member to be hidden in the casing.

7. The drug delivery device of claim 5, wherein, when the casing is placed on an object and the safety detection member faces the object, the object forces the safety detection member to be hidden in the casing.

8. The drug delivery device of claim 7, wherein, when the casing moves away from the object, the safety detection member is automatically exposed from the casing.

9. The drug delivery device of claim 5, wherein the safety detection member surrounds the injection member.

10. The drug delivery device of claim 9, wherein, when the casing moves away from the object, the safety detection member is automatically exposed from the casing and self-locked.

11. The drug delivery device of claim 5, wherein the sensing module comprises at least one optical sensor configured to sense an amount of light related to at least one of the drug infusion member, the injection member and the safety detection member.

12. The drug delivery device of claim 5, wherein the sensing module comprises an optical sensor, and the drug infusion member, the safety detection member and the injection member are arranged along an optical axis of the optical sensor.

13. The drug delivery device of claim 5, wherein the sensing module comprises two optical sensors, one of the drug infusion member, the safety detection member and the injection member is arranged along an optical axis of one of the two optical sensors, and the other two of the drug infusion member, the safety detection member and the injection member are arranged along an optical axis of the other one of the two optical sensors.

14. The drug delivery device of claim 1, further comprising a motor disposed in the casing, coupled to the processing unit and configured to infuse the medical fluid into the drug infusion member, wherein the sensing module is configured to sense a current number of revolutions of the motor; wherein, when the current number of revolutions is smaller than or equal to a first predetermined number of revolutions, the processing unit determines that the drug delivery device is in an injection-in-progress stage; wherein, when the current number of revolutions is larger than the first predetermined number of revolutions, the processing unit determines that the drug delivery device is in a ready-to-stop stage.

15. The drug delivery device of claim 14, wherein, when the drug delivery device is in the injection-in-progress stage and the sensing module senses that at least one air bubble exists in the drug infusion member, the processing unit determines that the drug infusion member is leaking.

16. The drug delivery device of claim 15, further comprising an alarm unit disposed in the casing and coupled to the processing unit, wherein, when the processing unit determines that the drug infusion member is leaking, the processing unit controls the alarm unit to send out an alarm.

17. The drug delivery device of claim 14, wherein, when the drug delivery device is in the ready-to-stop stage and the sensing module senses that at least one air bubble exists in the drug infusion member, the processing unit determines whether a percentage of the at least one air bubble to the medical fluid within a time period is larger than a threshold; wherein, when the percentage of the at least one air bubble to the medical fluid within the time period is larger than the threshold, the processing unit determines that an injection process of the drug delivery device has been completed and controls the motor to stop.

18. The drug delivery device of claim 17, wherein, when the percentage of the at least one air bubble to the medical fluid within the time period is smaller than or equal to the threshold, the motor continues to operate until the current number of revolutions reaches a second predetermined number of revolutions; the second predetermined number of revolutions is larger than the first predetermined number of revolutions.

19. The drug delivery device of claim 14, wherein the sensing module comprises two optical sensors, the drug infusion member is arranged along an optical axis of one of the two optical sensors, and a rotating portion of the motor is arranged along an optical axis of the other one of the two optical sensors.

20. A drug delivery device comprising:
a casing;
a drug infusion member disposed in the casing;
a motor disposed in the casing and configured to infuse a medical fluid into the drug infusion member;
a sensing module disposed in the casing and configured to sense a status of the drug infusion member and sense a current number of revolutions of the motor; and
a processing unit disposed in the casing and coupled to the motor and the sensing module;
wherein, when the current number of revolutions is larger than a first predetermined number of revolutions, the processing unit determines that the drug delivery device is in a ready-to-stop stage;
wherein, when the drug delivery device is in the ready-to-stop stage and the sensing module senses that at least one air bubble exists in the drug infusion member, the processing unit determines whether a percentage of the at least one air bubble to the medical fluid within a time period is larger than a threshold;
wherein, when the percentage of the at least one air bubble to the medical fluid within the time period is larger than the threshold, the processing unit determines that an injection process of the drug delivery device has been completed and controls the motor to stop.

21. The drug delivery device of claim 20, wherein, when the percentage of the at least one air bubble to the medical fluid within the time period is smaller than or equal to the threshold, the motor continues to operate until the current number of revolutions reaches a second predetermined number of revolutions; the second predetermined number of revolutions is larger than the first predetermined number of revolutions.

22. The drug delivery device of claim 20, wherein, when the current number of revolutions is smaller than or equal to the first predetermined number of revolutions, the processing unit determines that the drug delivery device is in an injection-in-progress stage; wherein, when the drug delivery device is in the injection-in-progress stage and the sensing module senses that the at least one air bubble exists in the drug infusion member, the processing unit determines that the drug infusion member is leaking.

23. The drug delivery device of claim 22, further comprising an alarm unit disposed in the casing and coupled to the processing unit, wherein, when the processing unit determines that the drug infusion member is leaking, the processing unit controls the alarm unit to send out an alarm.

24. The drug delivery device of claim 20, wherein the sensing module comprises two optical sensors, the drug infusion member is arranged along an optical axis of one of the two optical sensors, and a rotating portion of the motor is arranged along an optical axis of the other one of the two optical sensors.
